SEQUENCE LISTING (2 sequences)

SEQ ID NO: 1 - protein, 4 aa, Artificial
  Synthetic
TPKR

SEQ ID NO: 2 - protein, 6 aa, Artificial
  Synthetic
TPKRGY

(19) United States
(12) Patent Application Publication
(10) Pub. No.: US 2020/0345857 A1
(43) Pub. Date: Nov. 5, 2020

(54) PHOSPHORYLCHOLINE-TUFTSIN CONJUGATE FOR TREATING OCULAR INFLAMMATION

(71) Applicant: TARSIUS PHARMA LTD., Zichron Yaacov (IL)

(72) Inventors: Daphne HAIM-LANGFORD, Zichron Yaacov (IL); Zohar MILMAN, Kibbutz Tel-Yosef (IL)

(21) Appl. No.: 16/760,719

(22) PCT Filed: Nov. 1, 2018
(86) PCT No.: PCT/IL2018/051166

§ 371 Date: Apr. 30, 2020

Related U.S. Application Data
(60) Provisional application No. 62/580,817, filed on Nov. 2, 2017.

(51) Int. Cl. A61K 47/64; A61K 38/07; A61K 9/00; A61P 27/02; A61P 29/00; A61K 47/54

(52) U.S. Cl. CPC A61K 47/64; A61K 38/07; A61K 47/544; A61P 27/02; A61P 29/00; A61K 9/0048

(57) ABSTRACT

Methods of treating or preventing ocular inflammation in a subject in need thereof, and methods of reducing the dose of a steroid administered to a subject suffering from ocular inflammation, comprising administering to an eye of the subject a pharmaceutical composition a phosphorylcholine-tuftsin conjugate comprising at least one phosphorylcholine moiety or a derivative thereof and tuftsin or a derivative thereof are provided.

### CROSS REFERENCE TO RELATED APPLICATIONS

This application claims the benefit of priority of U.S. Provisional Patent Application No. 62/580,817, filed Nov. 2, 2017, the contents of which are incorporated herein by reference in their entirety.

### FIELD OF THE INVENTION

The present invention is directed to the field of ocular inflammation treatment.

### BACKGROUND OF THE INVENTION

Ocular inflammation, an inflammation of any part of the eye is one of the most common ocular diseases. Ocular inflammation actually refers to a wide range of inflammatory disease of the eye, one of them is uveitis. These diseases are prevalent in all age groups, and some are associated with systemic diseases such as Crohn's disease, Behcet disease, Juvenile idiopathic arthritis and others. The inflammation can also be associated with other common eye symptoms such as dry eye and dry macular degeneration. Several drugs also have the known side effect of causing uveitis and/or dry eye. The most common treatment for ocular inflammation, is steroids and specifically corticosteroids. However, these treatments have several known and sometimes severe side effects.

Tuftsin-PhosphorylCholine (TPC) is a novel bi-specific small molecule with immunomodulatory activities. Tuftsin (Thr-Lys-Pro-Arg) is a self natural immunomodulating peptide produced by enzymatic cleavage of the Fc-domain of the heavy chain of IgG in the spleen. Phosphorylcholine (PC) is a small zwitterionic molecule secreted by helminths which permits helminths to survive in the host inducing a situation of immune tolerance as well as on the surface of some bacteria and apoptotic cells. Subcutaneous (5 μg/mouse) and oral (50 μg/mouse and 250 μg/mouse) administration of TPC has shown remarkable immunomodulatory effects in three experimental mouse models of autoimmune diseases. Administration of TPC prevented glomerulonephritis onset in lupus prone mice, reduced colitis in mice with dextran sodium sulfate induced colitis and prevented joint damage in mice with collagen-induced arthritis. In the three models, TPC inhibited proinflammatory cytokine expression such as IL-6, IL-17, TNFα, IFNγ, increased anti-inflammatory IL-10, enhanced expansion of T and B regulatory cells, overall resulting in a reduction of disease severity and longer survival of mice.

Methods of treating ocular inflammation which do not rely on steroids are greatly needed. Additionally, formulations of TPC for direct administration to the eye, and with very low doses of the drug are greatly beneficial.

### SUMMARY OF THE INVENTION

The present invention provides methods of preventing or treating ocular inflammation in a subject in need thereof, and methods of reducing the dose of a steroid administered to a subject suffering from ocular inflammation comprising administering to an eye of a subject a pharmaceutical composition comprising a phosphorylcholine-tuftsin conjugate comprising at least one phosphorylcholine moiety or a derivative thereof and tuftsin or a derivative thereof.

According to a first aspect, there is provided a method for treating or preventing ocular inflammation in a subject in need thereof, the method comprising administering to an eye of the subject a pharmaceutical composition comprising a very low dose of a phosphorylcholine-tuftsin conjugate comprising at least one phosphorylcholine moiety or a derivative thereof and tuftsin or a derivative thereof.

According to another aspect, there is provided a method of reducing the dose of a steroid administered to a subject suffering from ocular inflammation, the method comprising administering to an eye of the subject a pharmaceutical composition comprising a phosphorylcholine-tuftsin conjugate comprising at least one phosphorylcholine moiety or a derivative thereof and tuftsin or a derivative thereof.

According to some embodiments, the phosphorylcholine moiety or a derivative thereof and the tuftsin or a derivative thereof are linked. According to some embodiments, the phosphorylcholine moiety or a derivative thereof and the tuftsin or a derivative thereof are separated by a spacer. According to some embodiments, the spacer is at least two amino acids. According to some embodiments, the spacer is Glycine-Tyrosine.

According to some embodiments, the treating comprises reducing inflammation. According to some embodiments, the reducing inflammation comprises reducing secretion of at least one pro-inflammatory cytokine in the eye of the subject. According to some embodiments, the pro-inflammatory cytokine is TNFα. According to some embodiments, the reducing inflammation comprises increasing secretion of at least one anti-inflammatory cytokine in the eye of the subject. According to some embodiments, the anti-inflammatory cytokine is IL-10.

According to some embodiments, reducing a dose of a steroid comprises reducing inflammation in the eye that is equal to or greater than a reduction in inflammation induced by a non-reduced dose of the steroid.

According to some embodiments, the method further comprising administering a steroid.

According to some embodiments, the ocular inflammation is uveitis. According to some embodiments, the ocular inflammation comprises dry eye, dry macular degeneration, and post operation inflammation.

According to some embodiments, the pharmaceutical composition is formulated for ocular administration. According to some embodiments, the formulated for ocular administration comprises any one of an eye drop formulation, an ointment formulation, and an injection formulation. According to some embodiments, the pharmaceutical composition comprises any one of a viscosity enhancer, a permeation enhancer or both. According to some embodiments, the pharmaceutical composition comprises a viscosity enhancer.

According to some embodiments, the very low dose is a dose at or below 0.005 μg/ml.

According to some embodiments, the steroid is a corticosteroid. According to some embodiments, the reduction in a dose of a steroid is at least a 10% reduction.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description together with the drawings makes apparent to those skilled in the art how embodiments of the invention may be practiced.

FIGS. 1A-1B: Very low dose TPC has a strong immunomodulatory effect. Bar graphs showing the effects of TPC at a range of doses, on anti-CD3-activated PBMCs 48 h after treatment. Secretion of pro-inflammatory cytokine TNFα (1A) and anti-inflammatory cytokine IL-10 (1B) are shown. Column statistics, one-sample t test compared to a hypothetical value of 100 was used. *—P<0.05, **—P<0.01, ***—P<0.005

FIG. 2: TPC enhances the anti-inflammatory effect of steroids. A bar graph showing the effect of IL-4/IL-13, TPC, dexamethasone and dexamethasone+TPC on IL-10 secretion by macrophages. *—P<0.05, **—P<0.01

### DETAILED DESCRIPTION OF THE INVENTION

The present invention, in some embodiments, provides methods of treating or preventing ocular inflammation in a subject in need thereof, and reducing the dose of a steroid administered to a subject suffering from ocular inflammation, the methods comprising administering to an eye of a subject a pharmaceutical composition comprising a very low dose of a phosphorylcholine-tuftsin conjugate comprising at least one phosphorylcholine moiety or a derivative thereof and tuftsin or a derivative thereof.

By a first aspect, there is provided a method for treating or preventing ocular inflammation in a subject in need thereof, the method comprising administering to the subject a pharmaceutical composition comprising a phosphorylcholine-tuftsin conjugate comprising at least one phosphorylcholine moiety or a derivative thereof and tuftsin or a derivative thereof.

By another aspect, there is provided a method of reducing a dose of a steroid administered to a subject suffering from ocular inflammation, the method comprising administering to the subject a pharmaceutical composition comprising a phosphorylcholine-tuftsin conjugate comprising at least one phosphorylcholine moiety or a derivative thereof and tuftsin.

The term “phosphorylcholine (PC) conjugate” as used herein, refers to a phosphorylcholine moiety or a derivative thereof linked to tuftsin (T), optionally via a spacer.

As used herein, the term “tuftsin” refers to a tetrapeptide (threonine-lysine-proline-arginine, TKPR; SEQ ID NO: 1). Tuftsin may be synthesized chemically or isolated from the spleen by enzymatic cleavage of the Fc domain of IgG heavy chain. Tuftsin is known for its phagocytosis-stimulating activity and augmentation of antigen presenting capacity of macrophages in-vitro and in-vivo. According to some embodiments, tuftsin may be considered as an immunomodulatory molecule.

The term “derivative of phosphorylcholine” as used herein, refers to any compound that is based off phosphorylcholine. The term “derivative of tuftsin” as used herein, refers to any polypeptide that is based off of TKPR. In some embodiments, the derivative retains the immunomodulatory effects of phosphorylcholine and/or tuftsin. In some embodiments, the derivative is a derivative comprising phosphorylcholine. In some embodiments, the derivative is a derivative comprising TKPR. A derivative is not merely a fragment of the polypeptide, nor does it have amino acids replaced or removed (an analog), rather it may have additional modification made to the polypeptide, such as a post-translational modification.

In some embodiments, the derivative of phosphorylcholine is selected from: 4-amino-phenyl-phosphocholine, 4-diazonio-phenyl-phosphorylcholine, 4-nitro-phenyl-phosphocholine and 12-(3-iodophenyl)dodecyl-phosphocholine among others. Each possibility is a separate embodiment of the invention.

The terms “tuftsin derivative”, “TD” and “tuftsin-derived carrier moiety” are interchangeable and refer to tuftsin (TKPR, SEQ ID NO: 1) attached to at least two additional amino acids which are independently selected. Non-natural amino acids, preferably non-charged and non-polar non-natural amino acids such as β-alanine-6-aminohexanoic acid and 5-aminopentanoic acid, may also be comprised in the tuftsin derivative. In some embodiments, the tuftsin derivative is Threonine-Lysine-Proline-Arginine-Glycine-Tyrosine (TKPRGY, SEQ ID NO: 2).

The term “moiety” as used herein refers to a part of a molecule, which lacks one or more atom(s) compared to the corresponding molecule. The term “moiety”, as used herein, further relates to a part of a molecule that may include either whole functional groups or parts of functional groups as substructures. The term “moiety” further means part of a molecule that exhibits a particular set of chemical and/or pharmacologic characteristics which are similar to the corresponding molecule.

The terms “linked” or “attached” as used herein refer to a bond between at least two molecules or moieties such that they are a single molecule. In some embodiments, the bond is a chemical bond. In some embodiments, the bond is a covalent bond. According to the principles of the present invention, the natural and non-natural amino-acids comprised in the tuftsin derivative are adjacent and attached to one another, while the at least one phosphorylcholine derivative is attached to the at least one tuftsin derivative either directly or indirectly via a spacer. In some embodiments, the at least one phosphorylcholine or derivative thereof is linked to the N-terminus of at least one tuftsin or derivative thereof. In some embodiments, the at least one phosphorylcholine or derivative thereof is linked to the C-terminus of at least one tuftsin or derivative thereof.

The term “spacer”, as used herein, refers to a connecting or otherwise bridging element between the tuftsin derivative and the PC derivative, typically linked by chemical methods or biological means thereto. Non-limiting examples of spacers include: amino acids, peptides, polypeptides, proteins, hydrocarbons and polymers among others. Each possibility is a separate embodiment of the invention. In some embodiments, the spacer is at least 2 amino acids. In some embodiments, the spacer is Glycine-Tyrosine. In some embodiments, the spacer is attached to the C-terminus of TKPR. In some embodiments, the spacer is attached to the N-terminus of TKPR.

In certain embodiments, the phosphorylcholine-tuftsin conjugate described above comprises one phosphorylcholine derivative attached to one tuftsin derivative. In certain embodiments, the phosphorylcholine-tuftsin conjugate described above comprises a plurality of phosphorylcholine derivatives attached to a plurality of tuftsin derivatives. In certain embodiments, the phosphorylcholine-tuftsin conjugate described above comprises a plurality of tuftsin derivatives attached to one phosphorylcholine derivative. In certain embodiments, the phosphorylcholine-tuftsin conjugate described above comprises a plurality of phosphorylcholine derivatives attached to one tuftsin derivative.

In certain embodiments, the phosphorylcholine-tuftsin conjugate described above comprises at least one phosphorylcholine or derivative thereof and the at least one tuftsin or derivative thereof separated by a spacer.

In some embodiments, the administering is to an eye of the subject. Ocular administration of a drug or composition is well known in the art. In some embodiments, ocular administration comprises dropping the composition on to the eye. In some embodiments, ocular administration comprises application to the eye, to the out surface of the eye, to the interior of the eye, to the blood vessels in contact with the eye, to the orbit, to the socket of the eye, to the epidermal surface and tissues that surround the eye, to the eyelid, to the eyelashes, and to the fatty deposits surrounding the eye. In some embodiments, a blood vessel in contact with the eye is selected from the ophthalmic artery, the central retinal artery, a posterior ciliary artery, and an anterior ciliary artery. In some embodiments, ocular administration comprises application to the eye, to the fluid around the eye, to the corner of the eye, to the tear ducts, to the anterior chamber of the eye, to the posterior chamber of the eye, to the choriod, to the retina, to the lense, to the uvea, or under the eye lids. Each possibility represents a separate embodiment of the invention.

As used herein, the term “pharmaceutical composition” refers to any composition comprising the phosphorylcholine conjugate and at least one other ingredient, as well as any product which results, directly or indirectly, from combination, complexation, or aggregation of any two or more of the ingredients, from dissociation of one or more of the ingredients, or from other types of reactions or interactions of one or more of the ingredients. Accordingly, the term “pharmaceutical composition” as used herein may encompass, inter alia, any composition made by admixing a pharmaceutically active amount of the conjugate and one or more pharmaceutically acceptable carriers. In some embodiments, the pharmaceutical composition comprises a pharmaceutically acceptable carrier, diluent or excipient.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition is formulated for ocular administration. Medicinal compostions for ocular administration are well know in the art and may comprise adjuvants, excipients or carriers specific for this purpose. Examples of such include but are not limited to, fluids at biological pH (6.5-7.5), preservatives, viscosity enhancers and permeation enhancers. In some embodiments, the pharmaceutical composition comprises a permeation enhancer, a viscosity enhancer or both. In some embodiments, the pharmaceutical composition comprises a viscosity enhancer. In some embodiments, a formulation for ocular administration comprises any one of an eye drop formulation, an ointment formulation, and an injection formulation.

As used herein, a “viscosity enhancer” refers to any substance that increases the viscosity of the solution to be administered to the eye. In some embodiments, the viscosity enhancer increases viscosity of an aqueous solution. A person skilled in the art will apresciated that increased viscosity improve residence time on the eye and increase bioavailability upon topical administration. Examples of viscosity enhancers include, but are not limited to hydroxy methyl cellulose, hydroxy ethyl cellulose, sodium carboxy methyl cellulose, hydroxypropyl methyl cellulose and polyalcohol.

As used herein, a “permeation enhancer” refers to any substance that improves corneal uptake by modifying corneal integrity and thus increase bioavailability in the eye. Examples of viscosity enhancers include, but are not limited to, benzalkonium chloride, polyoxyethylene glycol esters, polycarbophil-cysteine and cyclodextrins.

The term “therapeutically effective amount” refers to the amount of the conjugate effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition comprises a very low dose of the phosphorylcholine-tuftsin conjugate. In some embodiments, the pharmaceutical composition comprises at most 50 μg/ml, 5 μg/ml, 0.5 μg/ml, 0.05 μg/ml, 0.005 μg/ml, 0.0005 μg/ml, 0.00005 μg/ml, 0.000005 μg/ml, 0.0000005 μg/ml, 0.00000005 μg/ml TPC. Each possibility represents a separate embodiment of the invention. In some embodiments, a very low dose is a dose at or below 0.5, 0.05, 0.005, 0.0005, 0.00005, 0.000005, 0.0000005, or 0.00000005 μg/ml. Each possibility represents a separate embodiment of the invention. In some embodiments, the very low dose is a dose at or below 0.005 μg/ml. It will be understood that the direct administration of the drug to the site of inflammation may enhance the ability to use a very low dose and treat the inflammation. In some embodiments, the steroid sparing dose of TPC is a very low dose of TPC. In some embodiments, the steroid sparing dose is a higher dose than a very low dose.

In some embodiments, the dose of drug that reaches the site of inflammation is very low. In some embodiments, the dose that reaches the site of inflammation is at most 50 μg/ml, 5 μg/ml, 0.5 μg/ml, 0.05 μg/ml, 0.005 μg/ml, 0.0005 μg/ml, 0.00005 μg/ml, 0.000005 μg/ml, 0.0000005 μg/ml, 0.00000005 μg/ml TPC. Each possibility represents a separate embodiment of the invention. A person skilled in the art will appreciate that eye drops in particular, and to an extent ointments as well, will not perfectly reach the site of inflammation. As such the dose will need to be increased or decreased as determined by a skilled artisan to compensate for the mode of administration. Doses by intraocular injection will more directly reach the site of inflammation and again the dose administered will need to adjusted accordingly.

As used herein, the term “ocular inflammation” refers to any inflammation of any part of the eye. In some embodiments, the inflammation is of the middle layer of the eye. In some embodiments, the inflammation is uveitis. In some embodiments, the ocular inflammation comprises dry eye or dry macular degeneration. In some embodiments, the ocular inflammation is associated with another disease. Non-limiting examples of systemic diseases which can result in ocular inflammation are Crohn's disease, Behcet disease, Juvenile idiopathic arthritis. In some embodiments, the ocular inflammation is associated with an adverse reaction to a drug or environmental trigger. Non-limiting examples of such include Rifabutin, quinolones, vaccines and allergens. In some embodiments, the ocular inflammation is associated with post operation inflammation. Non-limiting examples of such include post-cataract surgery, laser eye surgery and corneal transplantation.

As used herein, the terms “treatment” or “treating” of ocular inflammation encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, treating ocular inflammation comprises at least one of preventing the onset of ocular inflammation, attenuating the progress of ocular inflammation and inhibiting the progression of ocular inflammation.

In some embodiments, treating comprises reducing inflammation. In some embodiments, treating comprises reducing abnormal inflammation. In some embodiments, treating comprises reducing inflammation in an eye of the subject. In some embodiments, treating comprises reducing intraocular pressure associated with ocular inflammation.

In some embodiments, the method of treating or preventing further comprises administering a steroid. In some embodiments, the steroid is a corticosteroid. In some embodiments, TPC and a steroid are administered together. In some embodiments, TPC and a steroid are administered concomitantly. In some embodiments, the TPC is administered first. In some embodiments, the steroid is administered first. In some embodiments, a very low dose of TPC is administered with the steroid.

In some embodiments, reducing a dose of a steroid comprises retaining the reduction in inflammation induced by the full dose of the steroid. In some embodiments, reducing a dose of a steroid comprises retaining the alleviation of symptoms induced by the full dose of the steroid. That is, though the steroid would be reduced the reduction in inflammation and/or alleviation of symptoms would not be reduced. In some embodiments, the reducing a dose of a steroid comprises reducing inflammation and/or symptoms in the eye that is equal to or greater than the reducing in inflammation induced by a non-reduced dose of the steroid. In some embodiments, the non-reduced dose is the full dose. In some embodiments, equal reduction in inflammation is brought about by increasing secretion of a pro-inflammatory steroid. In some embodiments, equal reduction in inflammation is brought about by decreasing secretion of a pro-inflammatory steroid and increasing or decreasing secretion of an anti-inflammatory steroid.

In some embodiments, treating comprises reducing secretion of at least one pro-inflammatory cytokine. In some embodiments, reducing inflammation comprises reducing secretion of at least one pro-inflammatory cytokine. In some embodiments, the secretion is in an eye of the subject. In some embodiments, treating comprises reducing secretion of a plurality of pro-inflammatory cytokines. In some embodiments, reducing inflammation comprises reducing secretion of a plurality of pro-inflammatory cytokines. In some embodiments, at least 1, 2, 3, 4, or 5 pro-inflammatory cytokines are reduced. Each possibility represents a separate embodiment of the invention. In some embodiments, treating comprises reducing the levels of at least one pro-inflammatory cytokine in the subject. In some embodiments, reducing inflammation comprises reducing the levels of at least one pro-inflammatory cytokine in the subject. In some embodiments, the levels are reduced in an eye. In some embodiments, the pro-inflammatory cytokine is TNFα. Other examples of pro-inflammation cytokines include, but are not limited to, IL-1, IL-1B, interferon gamma (IFNγ), IL-12, IL-18 and colony-stimulating factor 2 (CSF2).

In some embodiments, reducing inflammation comprises at least one of increasing secretion of at least one anti-inflammatory cytokine in the eye of the subject, decreasing secretion of at least one pro-inflammatory cytokine in the eye of the subject, increasing the number of Tregs in the eye of the subject and increasing the number of M2 macrophages in the eye of the subject. T regulatory cells (Tregs) are well known in the art and are known to have immunosuppressant effects and the ability to locally lower inflammation. M2 macrophages also are immunotolerant and secret anti-inflammatory cytokines.

In some embodiments, treating comprises increasing secretion of at least one anti-inflammatory cytokine. In some embodiments, reducing inflammation comprises increasing secretion of at least one anti-inflammatory cytokine. In some embodiments, the secretion is in an eye of the subject. In some embodiments, treating comprises increasing secretion of a plurality of anti-inflammatory cytokines. In some embodiments, reducing inflammation comprises increasing secretion of a plurality of anti-inflammatory cytokines. In some embodiments, at least 1, 2, 3, 4, or 5 anti-inflammatory cytokines are increased. Each possibility represents a separate embodiment of the invention. In some embodiments, the levels are increased in an eye of the subject. In some embodiments, the anti-inflammatory cytokine is IL-10. Other examples of anti-inflammation cytokines include, but are not limited to, IL-4, IL-13, IFNα and transforming growth factor beta (TGFβ).

In some embodiments, reducing comprises at least a 10%, 20%, 30%, 40%, 50%, 60%, 70%, 80%, 90%, 95%, 99% or 100% reduction. Each possibility represents a separate embodiment of the invention. It will be understood by one skilled in the art that each cytokine need not be reduced by the same amount. Some cytokines may be reduced by more than others.

In some embodiments, increasing comprises at least a 10%, 20%, 30%, 40%, 50%, 60%, 70%, 80%, 90%, 95%, 99%, 100%, 150%, 200%, 300%, 400%, 500%, 1000%, or 10000% increase. Each possibility represents a separate embodiment of the invention. It will be understood by one skilled in the art that each cytokine need not be increased by the same amount. Some cytokines may be increased by more than others.

Steroid dosing for treating ocular inflammation is well characterized in the art. Types of ocular inflammation may have a different dose of steroid, as is indicated in the art. According to the methods of the invention TPC may be used to decrease the dose of a steroid administered to a subject suffering from ocular inflammation. In some embodiments, the steroid is a corticosteroid. In some embodiments, the reduction in dose is at least a 5%, 10%, 15%, 20%, 25%, 30%, 35%, 40%, 45%, 50%, 55%, 60%, 65%, 70%, 75%, 80%, 85%, 90%, 95% or 100% reduction in the dose of corticosteroids. In some embodiments, the reduction in dose is a reduction in the frequency of dosing. It will be understood by one skilled in the art, that receiving steroids every other day when the dose had previously been administered daily would be considered a reduction in dose. Similarly, an intermittent dosing schedule that had been 10 days steroid/10 days without, that is changed to 10 days steroid/15 days without, or 5 days steroid/10 days without, or similar alterations, would also be considered a reduction in dose. Any reduction in the amount of steroid that the subject receives over a given period of time, is to be considered a reduction in dose.

In some embodiments, reduction in a dose of steroid is a reduction to zero. In some embodiments, reduction in a dose comprises no longer treating with steroids. In some embodiments, reduction in a dose occurs after a subject has already been treated with steroids. In some embodiments, reduction in a dose occurs before the subject has begun steroid therapy. In some embodiments, reduction in a dose occurs before the subject has begun any therapy. In some embodiments, reduction in a dose comprises reduction in a dosing regimen over time. In some embodiments, reduction in a dose comprises reduction in a dosing regimen earlier than the reduction would occur without the treatment of the invention.

As used herein, the terms “administering”, “administration”, and like terms refer to any method which, in sound medical practice, delivers a composition containing an active agent to a subject in such a manner as to provide a therapeutic effect. In some embodiments, the administering is ocular or intraocular.

According to other embodiments, the pharmaceutical composition is in the form of solution, suspension, eye drops, ointment, an intraocular injection among other types of pharmaceutical compositions. Each possibility is a separate embodiment of the invention.

By another aspect, there is provided a use of a pharmaceutical composition comprising a phosphorylcholine-tuftsin conjugate comprising at least one phosphorylcholine moiety or a derivative thereof and tuftsin or a derivative thereof for treating or preventing ocular inflammation. In some embodiments, the pharmaceutical composition comprises a very low dose of the phosphorylcholine-tuftsin conjugate.

By another aspect, there is provided a use of a phosphorylcholine-tuftsin conjugate comprising at least one phosphorylcholine moiety or a derivative thereof and tuftsin or a derivative thereof and a steroid for treating or preventing ocular inflammation. In some embodiments, the phosphorylcholine-tuftsin conjugate and the steroid are in one pharmaceutical composition. In some embodiments, the phosphorylcholine-tuftsin conjugate and the steroid are in separate compostions.

By another aspect, there is provided a use of a phosphorylcholine-tuftsin conjugate comprising at least one phosphorylcholine moiety or a derivative thereof and tuftsin or a derivative thereof for reducing a dose of a steroid administered to treat ocular inflammation.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, chemical, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1: Effects of TPC on PBMCs

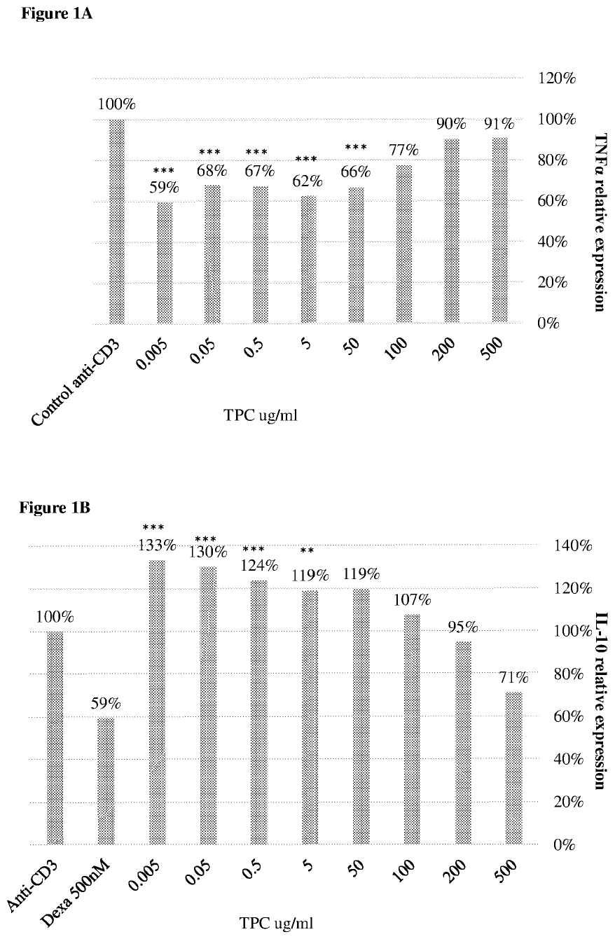

The immunomodulatory effect of TPC was determined in vitro on PBMCs from healthy donors. PBMCs were harvested and allowed to adhere to culture plates. TPC at various concentrations, or PBS or dexamethasone as controls were then added and an hour later anti-CD3 stimulating antibody was added to induce an inflammatory state. 48 hours later the supernatant was harvested from the cells and cytokine levels were assayed using standard Luminex protocols. Levels of cytokine induction was interpolated from standard curves, using 5-parameter non-linear regression analyses, where y=(A+((B−A)/(1+(((B−E)/(E−A))*((x/C)\hat{ }D))))). The interpolated data was then normalized to vehicle controls (anti-CD3 stimulation alone). TPC treatment decreased expression of the pro-inflammatory cytokine TNFα, however it was notable that the greatest reduction was seen at very low doses (FIG. 1A). At a dose as low as 0.005 μg/ml the TPC showed its greatest effectiveness, lowering TNFα levels by 41%. TPC treatment also increased expression of the anti-inflammatory cytokine IL-10 (FIG. 1B). Once again, the effect was greatest at very low doses, as the 0.005 μg/ml TPC dose had the strongest effect causing a 33% increase in IL-10 levels. Effects of TPC on PBMC cytokine secretion were equal if not superior to dexamethasone, as the steroid had a negative effect on IL-10 levels. (FIG. 1B). Indeed, as compared to dexamethasone, TPC had a 220% increase on IL-10 secretion. This suggests that supplementing a dexamethasone dose with TPC will have a beneficial effect on inflammation reduction.

### Example 2: TPC can Reduce Steroid Dose

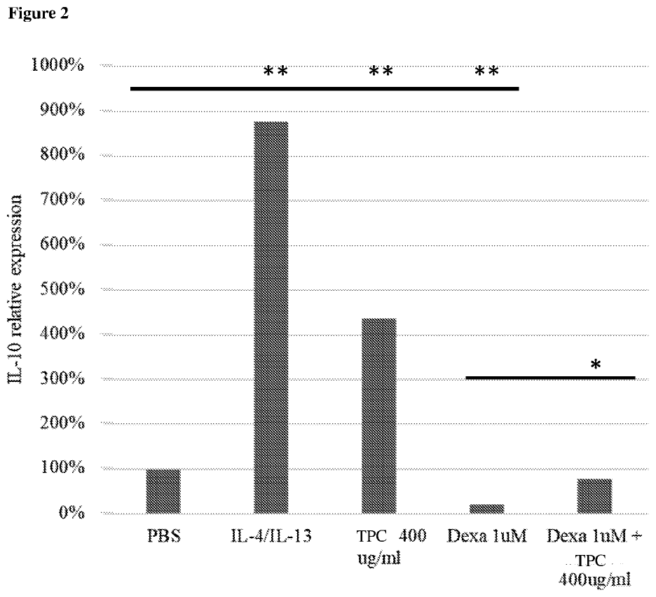

To test the hypothesis that a combination of steroids and TPC will have a beneficial effect on treating inflammation, cells of the THP-1 human monocyte cell line were differentiated to M0 macrophages with 100 ng/ml PMA for 48 hours and then polarized to M1 macrophages with 10 ng/ml LPS. These cells were then tested with the following compounds: PBS (negative control), IL-4 and IL-13 (20 ng/ml, positive control as they induce the cells to a M2 phenotype), TPC (400 ug/ml), dexamethasone (1 uM), and a combination of dexamethasone (1 uM) and TPC (400 ug/ml). IL-4 and IL-13 induced robust expression of IL-10 (FIG. 2). TPC also induced a strong increase in IL-10 expression while dexamethasone actually decreased expression of the anti-inflammatory cytokine. Importantly, co-treatment of dexamethasone and TPC improved IL-10 expression levels back to that of control levels, ameliorating the negative effect of dexamethasone. This suggests that addition of TPC to steroid treatment is able to reverse the negative effects of the steroid on anti-inflammatory cytokine secretion. Further, since TPC itself reduces pro-inflammatory cytokine secretion as well, it may be used, not just in combination, but to reduce the total dose of steroid administered.

### Example 3: Lowest TPC Dose to Reduce Steroid Dose

PBMCs are harvested, plated, and stimulated as before. Increasingly lower doses of TPC are added to the cells and the effects on pro and/or anti-inflammatory cytokines and/or Treg and M1/M2 macrophage numbers are evaluated. A minimal effective dose is determined based on the lowest TPC concentration that can be administered and still increase the expression of anti-inflammatory cytokines (IL-10 at least), increase Treg/M2 macrophage number, and/or decrease the secretion of pro-inflammatory cytokines (TNFα at least). A dose of TPC is also combined with increasingly lower doses of dexamethasone, or another steroid, and the ability of the combined treatment to increase the expression of anti-inflammatory cytokines (IL-10 at least), increase Treg/M2 macrophage number, and/or decrease the secretion of pro-inflammatory cytokines (TNFα at least) is measured.

### Example 4: Ex-Vivo Effect on Ocular Immune Cells

Aqueous humor samples are acquired from patients afflicted with active non-infectious uveitis or other ocular inflammation, that undergo diagnostic or therapeutic paracentesis or cataract surgery. The samples are centrifuged, and the resultant cell pellet is re-suspended in culture medium. Equal numbers of cells are incubated with and without various concentrations of TPC, dexamethasone or another steroid is used as a positive control and cells may be activated as a negative control. As above the lowest effective dose is determined. As above combinations of TPC and decreasing concentrations of dexamethasone are monitored for their ability to decrease and/or maintain reduced inflammation. The production of cytokines, chemokines and Treg/macrophage is measured (at least IL-10 and/or TNFα).

### Example 5: In-Vivo Effect

An animal model of uveitis or any other inflammatory/autoimmune ocular condition is employed to test the in vivo effect of low dose TPC and/or the ability of TPC to reduce steroid dose. For uveitis, experimental autoimmune uveitis is induced in mice, rats, rabbits and/or monkeys by immunization with retinal antigens (arrestin, inter-photoreceptor retinoid-binding protein, rhodopsin, opsin, recoverin, phosducin or similar). Pertussis toxin or tuberculosis bacteria may be used as an adjuvant for induction of the disease. After induction of the disease/condition, the animals are dosed with TPC at varying doses, steroid (dexamethasone or other) alone, and TPC with decreasing doses of steroid. The animals are monitored for ocular inflammation and clinical symptoms and aqueous humor, plasma samples and/or other tissues are extracted to examine the in vivo effect.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for treating or preventing ocular inflammation in a subject in need thereof, the method comprising administering to an eye of said subject a pharmaceutical composition comprising a very low dose of a phosphorylcholine-tuftsin conjugate comprising at least one phosphorylcholine moiety or a derivative thereof and tuftsin or a derivative thereof.

2. A method of reducing a dose of a steroid administered to a subject suffering from ocular inflammation, the method comprising administering to an eye of said subject a pharmaceutical composition comprising a phosphorylcholine-tuftsin conjugate comprising at least one phosphorylcholine moiety or a derivative thereof and tuftsin or a derivative thereof.

3. The method of claim 1, wherein said phosphorylcholine moiety or a derivative thereof and said tuftsin or a derivative thereof are linked.

4. The method of claim 2, wherein said phosphorylcholine moiety or a derivative thereof and said tuftsin or a derivative thereof are separated by a spacer.

5. The method of claim 4, wherein said spacer is at least two amino acids.

6. The method of claim 4, wherein said spacer is Glycine-Tyrosine.

7. (canceled)

8. The method of claim 2, wherein said reducing a dose of a steroid comprises reducing inflammation in said eye that is equal to or greater than a reduction in inflammation induced by a non-reduced dose of said steroid.

9. The method of claim 7, wherein said reducing inflammation comprises reducing secretion of at least one pro-inflammatory cytokine in said eye of said subject.

10. The method of claim 9, wherein said pro-inflammatory cytokine is TNFα.

11. The method of claim 8, wherein said reducing inflammation comprises increasing secretion of at least one anti-inflammatory cytokine in said eye of said subject.

12. The method of claim 11, wherein said anti-inflammatory cytokine is IL-10.

13. (canceled)

14. The method of claim 2, wherein said ocular inflammation is uveitis.

15. The method of claim 2, wherein said ocular inflammation comprises dry eye, dry macular degeneration, and post operation inflammation.

16. The method of claim 2, wherein said pharmaceutical composition is formulated for ocular administration.

17. The method of claim 16, wherein said formulated for ocular administration comprises any one of an eye drop formulation, an ointment formulation, and an injection formulation.

18. The method of claim 2, wherein said pharmaceutical composition comprises any one of a viscosity enhancer, a permeation enhancer or both.

19. The method of claim 18, wherein said pharmaceutical composition comprises a viscosity enhancer.

20. The method of claim 2, wherein said very low dose is a dose at or below 0.005 μg/ml.

21. The method of claim 2, wherein said steroid is a corticosteroid.

22. The method of claim 2, wherein the reduction in a dose of a steroid is at least a 10% reduction.
